(19) United States
(12) Patent Application Publication
(10) Pub. No.: US 2009/0240279 A1
(43) Pub. Date: Sep. 24, 2009

(54) Knitted Compressive Orthosis of the Lower Limb for Treating Chronic Venous Insufficiency

(76) Inventors: Francois Becker, Chamonix (FR); Francois Cros, Ivry Sur Seine (FR); Jose-Maria Fullana, Paris (FR)

Correspondence Address: JACOBSON HOLMAN PLLC, 400 SEVENTH STREET N.W., SUITE 600, WASHINGTON, DC, 20004, US

(21) Appl. No.: 11/922,347

(22) PCT Filed: Jun. 7, 2006
(86) PCT No.: PCT/FR2006/001284

§ 371 Date: Apr. 9, 2008

(30) Foreign Application Priority Data
Jun. 16, 2005 (FR) 05/06107

(51) Int. Cl. A61B 17/00

(52) U.S. Cl. 606/201

(57) ABSTRACT

This orthosis comprises a compressive leg portion extending upwards from the ankle. The structure of the row of stitches ensures a uniform distribution of the resilient return forces at the circumference of the orthosis around an outline corresponding to a horizontal section of the limb. The stitch structure and the dimensioning of the leg portion are selected in such a manner as to apply on a region (R) extending between the ankle and the knee, a therapeutic pressure once the orthosis has been put on the limb. The mean pressure Pc exerted on the center third (RC) of said compression region is greater both than the mean pressure Pb exerted on the distal third (RD) and than the mean pressure Pd exerted on the proximal third (RP) of said region, Pc, Pb, and Pd being the pressures exerted at the respective heights of points c, b, and d of the Hohenstein model.

The invention relates to compressive orthoses for the lower limbs, generally known as “compression stockings” that are indicated for various sets of symptoms of venous insufficiency of the lower limbs.

Although the term “stocking” is used below, the invention is not limited to one particular type of article, but applies equally to other forms of compression orthoses for one or both lower limbs, whether in the form of tights, single-legged tights, stockings, or socks.

Such orthoses are designed to produce a therapeutic effect by compressing the lower limb over a greater or lesser extent covering at least the region of the calf. This compression also presents a compression profile that is degressive going upwards from the ankle.

Because of their therapeutic purpose, such orthoses differ in particular from the stockings or socks proposed in U.S. Pat. No. 6,216,495 (Couzan et al.) that do not set out to treat pathologies such as chronic venous insufficiency (CVI) but that presents a pressure gradient along the leg adapted to the physiology of athletes with the pressure that is exerted on the region of the foot and of the ankle bone being less than the pressure exerted in the region of the calf, and pressure on the calf that is “downwardly degressive”, i.e. with a profile that is the opposite of the usual profile for therapeutic stockings for treating venous insufficiency.

Compression orthoses for therapeutic purposes are classified in the ASQUAL system into four compression classes, namely class I (13 hectopascals (hPa) to 20 hPa ≈10 millimeters of mercury (mmHg) to 15 mmHg at the ankle), class II (20 hPa to 27 hPa≈15 mmHg to 20 mmHg), class III (27 hPa to 48 hPa≈20 mmHg to 36 mmHg), and class IV (>48 hPa≈>36 mmHg).

The study of venous physiology, in particular using recent tools for modeling and stimulating compression, shows that the effectiveness of compression resides above all in improving the efficiency of the muscolo-aponeurotic pump of the calf. The magnitude of the “muscular pump” or “muscolo-aponeurotic pump of the calf” effect is described in particular in terms of the return venous blood flow, where physiological muscle contractions of the calf expel venous blood centripetally to the muscular and deep venous network, with chronic venous insufficiency being characterized by a failure of this muscular pump effect.

EP-A-1 240 880 (Stolk) proposes for this purpose a compression orthosis comprising in the region of the calf a zone that presents non-uniform relative elasticity along an outline at a given height, and in particular with a zone of elasticity that is locally small or zero (i.e. that is less deformable or not deformable) in the posterior face in the region of the calf, suitable for exerting an extra compression pressure on the lower limb in this region. The purpose is to exert compression on the calf mainly where such pressure is effective, i.e. against the rear face, and not in a manner that is uniform in all directions in a horizontal plane.

In practice, such an orthosis is found to be technically very difficult to fabricate, and in addition, in use, it is difficult to position the zone of low or zero elasticity exactly over the region of the bulge of the calf, and to keep it durably in position, the orthosis tending to slide relative to the limb under the combined effect of the movement of the patient and of the pressure exerted by the orthosis on the leg.

U.S. Pat. No. 3,386,270 (Simmons) describes a compression orthosis which, unlike the above, seeks to produce pressure that is as uniform as possible around an outline in a horizontal plane, in particular at the level of the calf. For that purpose, the orthosis includes in its posterior face in the region of the calf, a zone that includes a greater length of elastic yarn, and that is therefore locally more deformable, thus making it possible to compensate for the greater deformation that results from the volume of the calf which would otherwise have generated in this region extra compression pressure.

One of the objects of the invention is to propose means enabling the efficiency of the muscolo-aponeurotic pump of the calf to be improved in the context of chronic venous insufficiency pathology, by means of a compressive orthosis that is better adapted to this function than are the orthoses that have been proposed until now.

The starting point of the invention lies in the observation that, when suffering from minor or even moderate venous insufficiency without trophic disorders, it has not been shown that there is any need to exert external pressure on the ankle, other than in order to comply with the “principle” of degressivity.

That prejudice, whereby the stocking needs to exert maximum pressure at the ankle, and then to exert pressure that is degressive from the ankle to the calf or to the thigh, is based on the idea that in an orthostatic situation intravenous pressure is degressive from the ankle to the calf or to the thigh, and that chronic venous insufficiency begins with a reduction in the caliber or bore-size of surface or deep veins under the effect of the elastic compression exerted by the stocking. However, a study of venous physiology, in particular using recent tools for modeling and stimulating compression, shows that the effectiveness of compression lies rather in improving the efficiency of the muscolo-aponeurotic pump of the calf.

The invention proposes an orthosis of the general type disclosed in particular by above-mentioned U.S. Pat. No. 3,386,270 (Simmons), i.e. a compressive orthosis for the lower limb in the form of a knitted article of the stocking, sock, or tights type, comprising a leg portion that extends upwards from the ankle, said leg portion being a compressive portion in which the stitch structure and the dimensioning are selected in such a manner as that once the orthosis has been put on the limb it applies a therapeutic compression pressure over a compression region of the leg extending between the ankle and the knee, and it distributes resilient return forces uniformly over its circumference around an outline corresponding to a horizontal section of the limb.

In a manner characteristic of the invention, the stitch structure and the dimensioning of the leg portion are additionally selected in such a manner that, once the orthosis has been put on the limb: the mean pressure Pc exerted on the center third of said compression region is greater both than the mean pressure Pb exerted on the distal third of the compression region and than the mean pressure Pd exerted on the proximal third of the compression region. The mean pressure is the mean of the compression pressures exerted locally around an outline at a given height up the limb; and where Pc, Pb, and Pd are the pressures exerted at the heights of the respective points c, b, and d of the Hohenstein model.

The orthosis of the invention makes it possible in particular to exert compression mainly at the level where it is effective, i.e. at the level of the calf, and not in degressive manner with a maximum pressure at the ankle.

In addition, both for patient comfort and for the therapeutic effectiveness of the orthosis, it is appropriate to avoid any tourniquet effect below the knee that would oppose the improvement to the muscular pump effect.

The above-described orthosis is well adapted as an indication for minor or moderate chronic venous insufficiency without trophic troubles.

Advantageously, the stitch structure and the dimensioning of the leg portion can be selected in such a manner that:
the mean pressure Pc exerted on the central third lies in the range 1.5 to 2.5 times the mean pressure Pb exerted on the distal third;
the mean pressure Pd exerted on the proximal third is less than the mean pressure Pb exerted on the distal third;
the mean pressure Pc is greater than or equal to 20 mmHg; and
the mean pressure Pd is less than 15 mmHg.

There follows a description of an embodiment of a device with reference to the accompanying drawing.

The sole accompanying FIGURE shows a series of pressure profiles, respectively for conventional compression stockings of classes I to III, and for a stocking of the invention, in association with different levels of the leg at which the applied compression pressures are measured.

The orthesis of the invention is an orthesis of the sock, stocking, or tights type, comprising a leg portion extending upwards from the ankle and including the ankle region. The leg portion is a compressive portion in which the stitch structure and dimensions are selected so as to apply a compression pressure on the leg once the orthosis has been put onto the limb.

The stitch may be of the same type as those in conventional compression stockings, for example of the weft-lock, stocking, pinched or float micromesh, etc. type, all stitches that are themselves known to specialists in knitting techniques. The yarn selected for knitting this orthosis may be non-covered elastane, or elastane covered in cotton and polyamide, or elastane covered in polyamide without cotton, or indeed a mixture of elastane and of elasto-diene (synthetic rubber latex).

Under the effect of being put on the limb, the tensioned textile exerts compression resulting from the return force from the elastic fibers making up the material. For a given dimension of the article in the non-tensioned state, i.e. for a given circular section, with a material that presents a high (or “reinforced”) coefficient of elasticity, putting the orthosis onto the limb produces a stronger return force, and thus greater compression that is manifested locally by a higher normal component of pressure exerted on the surface of the limb.

The stitch and the yarn, and also the dimensions of the various rows of stitches, are defined in such a manner as to apply predetermined pressures at different heights up the leg, for example at ankle height, at the beginning of the calf, over the calf, at the back of the knee, etc. up to the top of the thigh with a thigh stocking or tights. These various pressures are defined for each compression class with reference to metrological templates such as the Hohenstein model.

For a given height, the structure of the row of stitches ensures that elastic return forces are distributed uniformly over the circumference of the stocking, i.e. along an outline corresponding to a horizontal section of the limb. The application of these resilient return forces on the outlined perimeter generates at a given point, in application of the Laplace equation, a local pressure that is inversely proportional to the radius of curvature of the outline at that point.

In the present description, the term “mean pressure” is used to designate the mean of the compression pressures exerted locally around an outline, at a given height up the limb.

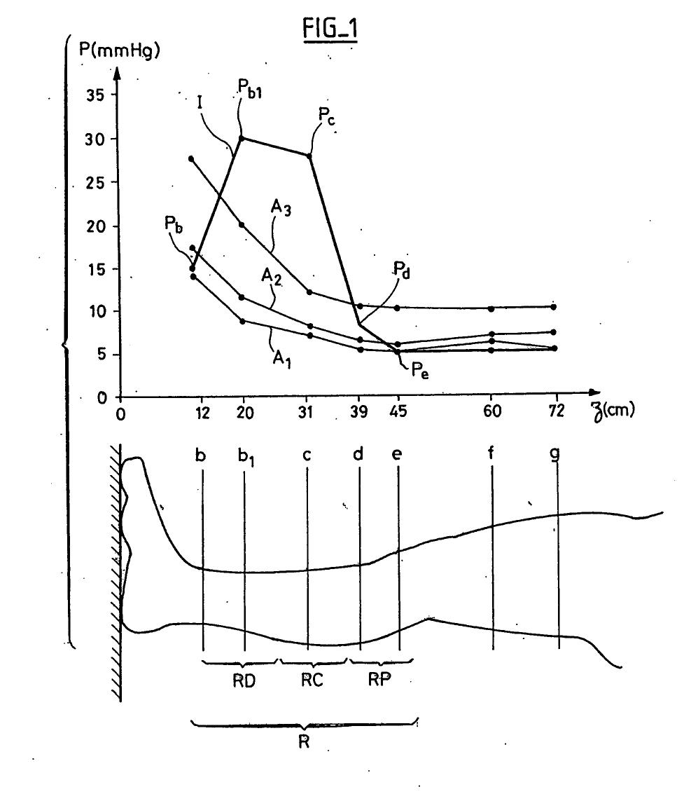

FIG. 1 shows measured values of mean pressure at various heights up the leg as defined by the Hohenstein model for a reference leg, namely:

b: at the minimum perimeter of the ankle, i.e. at a height z=12 cm above the ground;

b1: at the beginning of the Achilles tendon at z=20 cm;

c: at the maximum perimeter of the calf, at z=31 cm;

d: under the knee at z=39 cm;

e: at the back of the knee, at z=45 cm;

f: in the middle of the thigh, at z=60 cm; and

g: at the top of the thigh, at z=72 cm.

With conventional orthoses, the pressure exerted at the height of point b in the Hohenstein model, i.e. at the level of the ankle, is the pressure prescribed for the selected compression class (I, II, III, or IV), this pressure also corresponding to the value of the (theoretically) highest pressure exerted on the limb.

In the FIGURE, the characteristic shows the pressure profile measured for orthoses of class I (profile A1), of class II (profile A2), and of class III (profile A3), and for an orthosis of the invention (profile I).

These profiles are defined from pressure values measured on a reference template, which pressures can be sensed by means of sensors that are themselves conventional (sensors of Salzmann or other type) for measuring the pressures produced locally by the orthosis and delivering an electrical signal that can subsequently be converted and processed for various calculations and displays.

The pressures exerted at the level of the foot, i.e. at heights below 12 cm, are not considered as meaningful in the context of describing the present invention, which applies only to the pressure profile exerted on the limb from the ankle and above.

As can be seen in the FIGURE, conventional stockings deliver a profile that is degressive starting from the ankle (point b, altitude z=12 cm), with the highest pressure being exerted by the orthosis on the limb at this location.

The invention proposes a novel pressure profile which, in contrast, begins by being progressive up to the calf, and then degressive above the calf and going towards the knee.

Essentially, the region R where the compression is applied extends from the point b (ankle) to the point e (below the knee), and comprises a distal third RD, a central third RC, and a proximal third RP (the “thirds” not necessarily being of equal size, but corresponding to three successive zones in the region R).

The stocking is designed to exert maximum compression, no longer at the level of the ankle in the distal region RD, but in the central region RC at the level of the calf.

The distal region, which includes the ankle, is thus subjected to a pressure that is much lower than with a conventional stocking (e.g. a pressure Pb=15 mmHg at most), thus greatly improving ease with which the stocking can be put on and comfort at the level of the ankle and of the foot.

At the opposite end of the region R, the pressure in the proximal region RP is likewise a low pressure, so as to deliver in this region a pressure that is close to that delivered by a non-medical textile, for example lying in the range 5 mmHg to 10 mmHg, thus serving merely to hold the orthosis in place but without discomforting the patient in any way, in particular without producing any tourniquet effect at this level.

The stitch structure and the dimensioning of the leg portion may be selected in particular in such a manner that once the orthosis has been put on the limb, the mean pressure at point c lies in the range 1.5 to 2.5 times the mean pressure exerted at point b, the mean pressure at point c preferably being greater than or equal to 20 mmHg, and the pressure at point d less than 15 mmHg.

In the example of the progressive-degressive profile shown, the pressures measured at the successive heights b, b1, c, d, and e are as follows:

Pb=15 mmHg-whereas for conventional stockings the pressure at this level may be as much as 36 mmHg for stockings of class III complying with regulation prescriptions;

Pb1=30 mmHg;

Pc=28 mmHg;

Pd=8 mmHg;

Pe=5 mmHg.

It can thus be seen that compression pressure increases very quickly with height, which pressure reaches a maximum in a plateau occupying the calf region (points b1 and c), and then decreases very rapidly in the proximal region down to a value that is very small and not therapeutic, less than the pressures generally delivered by conventional stockings. Comfort is thus also increased at this level.

These low pressures both at the ankle and beneath the knee are likely to improve considerably compliance with a prescription for elastic venous compression, and thus the therapeutic effectiveness of the treatment in the long term.

1. A compression orthosis for the lower limb in the form of a knitted article of the stocking, sock, or tights type, comprising a leg portion that extends upwards from the ankle, said leg portion being a compressive portion having a stitch structure and dimensions that are selected so as:
once the orthosis has been put on the limb, to apply a therapeutic compression pressure over a compression region (R) of the leg extending between the ankle and the knee; and
to distribute resilient return forces uniformly over the circumference of the orthosis around an outline corresponding to a horizontal section of the limb;
the orthosis being characterized in that the stitch structure and the dimensioning of the leg portion are additionally selected in such a manner that, once the orthosis has been put on the limb:
the mean pressure Pc exerted on the center third (RC) of said compression region is greater both than the mean pressure Pb exerted on the distal third (RD) of the compression region and than the mean pressure Pd exerted on the proximal third (RP) of the compression region;
where mean pressure is the mean of the compression pressures exerted locally around an outline at a given height up the limb; and
where Pc, Pb, and Pd are the pressures exerted at the heights of the respective points c, b, and d of the Hohenstein model.

2. The orthosis of claim 1, in which the stitch structure and the dimensioning of the leg portion are chosen in such a manner that, once the orthosis has been put on the limb, the mean pressure Pc exerted on the center third (RC) of the compression region lies in the range 1.5 to 2.5 times the mean pressure Pb exerted on the distal third (RD) of the compression region.

3. The orthosis of claim 1, in which the stitch structure and the dimensioning of the leg portion are chosen in such a manner that, once the orthosis has been put on the limb, the mean pressure Pd exerted on the proximal third (RP) of the compression region is less than the mean pressure Pb exerted on the distal third (RD) of the compression region.

4. The orthosis of claim 1, in which the stitch structure and the dimensioning of the leg portion are chosen in such a manner that, once the orthosis has been put on the limb, the mean pressure Pc exerted on the center third (RC) of the compression region is greater than or equal to 20 mmHg.

5. The orthosis of claim 1, in which the stitch structure and the dimensioning of the leg portion are chosen in such a manner that, once the orthosis has been put on the limb, the mean pressure Pd exerted on the proximal third (RP) of the compression region is less than 15 mmHg.
